(19) United States
(12) Patent Application Publication
(10) Pub. No.: US 2018/0078346 A1
(43) Pub. Date: Mar. 22, 2018

(54) DENTAL MEMBRANE

(71) Applicant: AMOLIFESCIENCE CO., LTD., Seoul (KR)

(72) Inventors: In Yong SEO, Seoul (KR); Seung Hoon LEE, Paju-si (KR); Song Hee KOO, Seoul (KR); Ji Hyun LEE, Incheon (KR)

(21) Appl. No.: 15/561,763

(22) PCT Filed: May 19, 2016
(86) PCT No.: PCT/KR2016/005294

§ 371 Date: Sep. 26, 2017

(30) Foreign Application Priority Data
Jun. 1, 2015 (KR) 10-2015-0077317

(51) Int. Cl. A61C 8/02; A61L 31/14; A61L 31/06; A61L 31/04

(52) U.S. Cl. CPC A61C 8/0006; A61L 31/148; A61L 31/146; A61L 31/048; A61L 31/06

(57) ABSTRACT

Provided is a dental membrane which includes: a first support made by accumulating first nanofibers of a biodegradable polymer obtained by electrospinning and having a plurality of pores formed therein; a second support made by accumulating second nanofibers of a biodegradable polymer obtained by electrospinning on the first support, and having a plurality of pores formed therein, in which the second nanofibers have diameters larger than the diameters of the first nanofibers; and a third support made by accumulating third nanofibers of a biodegradable polymer obtained by electrospinning on the second support, and having a plurality of pores formed therein, in which the third nanofibers have diameters smaller than the diameters of the second nanofibers.

### TECHNICAL FIELD

The present invention relates to a dental membrane, and more particularly, to a dental membrane capable of controlling a decomposition rate of a laminated support thereby maximizing function of the dental membrane and improving skin adhesion.

### BACKGROUND ART

There are 32 adult teeth, which may be lost by accidents, or may lose their lives due to genetically poor teeth, or aging and bad habits. Treatment operations of replacing lost teeth have been made.

In the past, artificial crowns or bridges, and interlocking partial dentures or whole dentures have been used to restore the tooth's ability when the teeth fail to function due to missing or poor teeth. However, there was a problem of shaving the healthy tooth on the side to secure such substitute.

In order to solve this problem, an implant treatment that can be fixed without moving like a denture and can have a feel like his or her own teeth has become popular recently.

A dental implant is a device that provides a healthy oral cavity by implanting an artificial tooth that can replace a lost tooth. The dental implant not only does not injure the surrounding teeth, but also has the same function and shape as a natural tooth, and does not cause cavity, to thus be used semi-permanently and cause an increase in use.

Generally, a dental implant consists of a dental implant crown that serves as a tooth and a fixture that is embedded in the alveolar bone and that serves as a root of the tooth. Here, an abutment for supporting the dental implant crown is further provided, and a screw for combining the abutment and the fixture is added.

Meanwhile, the dental membrane is a dental shielding membrane that can be implanted into injured bones or periodontal tissues during implant or periodontal surgery, to regenerate bones and periodontal tissues, thereby preventing penetration of adjacent soft tissues or undesirable cells, and has recently been used in dental procedures. Efforts are needed to diversify and improve the functions of such dental membranes.

Korean Patent Laid-open Publication No. 2014-0111256 discloses a flexible dental surgical membrane including: a chitosan membrane having a thickness in the range of about 100 microns to about 0.5 mm; and pores that allow the chitosan membrane to have permeability to oxygen in the atmosphere and normal human red blood cells in the environment of the human oral cavity, wherein the chitosan has a molecular weight of at least 400,000 daltons.

This membrane for dental surgery is a membrane having pores obtained by applying a slurry in which porogen particles and chitosan are dispersed in an acidic aqueous solution to the surface of a support, evaporating the acidic aqueous solution from the applied slurry, and removing the porogen particles with a solvent, and does not have a structure similar to that of a human cell tissue. Therefore, there is a defect that skin adhesion can be deteriorated.

### DISCLOSURE

### Technical Problem

The present invention has been made in view of the above-mentioned defect, and its object is to provide a dental membrane which is implemented with a nanofiber web of biodegradable polymer which is harmless to the human body and which does not require a separate operation for removing the implanted membrane.

Another object of the present invention is to provide a dental membrane capable of regulating the rate of decomposition of a support made of a biodegradable polymer with diameters of nanofibers forming the support or thickness of the support, thereby maximizing the function of the dental membrane.

Another object of the present invention is to provide a dental membrane capable of improving skin adhesion by forming a membrane with a nanofiber web having a structure most similar to an extracellular matrix of the human body.

### Technical Solution

According to an aspect of the present invention, there is provided a dental membrane comprising: a first support made by accumulating first nanofibers of a biodegradable polymer obtained by electrospinning and having a plurality of pores formed therein; a second support made by accumulating second nanofibers of a biodegradable polymer obtained by electrospinning on the first support, and having a plurality of pores formed therein, in which the second nanofibers have diameters larger than the diameters of the first nanofibers; and a third support made by accumulating third nanofibers of a biodegradable polymer obtained by electrospinning on the second support, and having a plurality of pores formed therein, in which the third nanofibers have diameters smaller than the diameters of the second nanofibers.

In the dental membrane according to an embodiment of the present invention, the diameters in size of the third nanofiber of the third support may be smaller than the diameters in size of the first nanofiber of the first support.

In the dental membrane according to an embodiment of the present invention, the diameters of the first nanofibers may be less than 100 nm.

In the dental membrane according to an embodiment of the present invention, the diameters of the third nanofibers may be in the range of 200 nm to 1 μm.

In the dental membrane according to an embodiment of the present invention, the biodegradable polymer may be one or a mixture of at least two of PLA (Poly Lactic Acid), PLLA (Poly(L-lactic acid)), PGA (Poly(glycolic acid)), PLGA (Poly(lactide-co-glycolide)), PCL (Polycaprolactone) and PDO (1,3-Propanediol).

In the dental membrane according to an embodiment of the present invention, at least one of the first to third nanofibers may include a hydrophilic agent.

In the dental membrane according to an embodiment of the present invention, the hydrophilic agent may be one of Tween 80, Pluronic and PVP.

In the dental membrane according to an embodiment of the present invention, the first support may be in contact with a living tissue to be grown, the thickness of the second support may be thicker than the thickness of the first support, and the thickness of the third support may be thicker than the thicknesses of the first and second supports.

Here, the first support may have a thickness capable of being decomposed in 1 to 2 months, the second support may have a thickness capable of being decomposed in 3 to 4 months, and the third support may have a thickness capable of being decomposed in 5 to 6 months.

According to another aspect of the present invention, there is provided a dental membrane comprising: a first support made by accumulating nanofibers containing biodegradable polymers obtained by electrospinning and bone growth factors and having a plurality of pores formed therein; a second support made by accumulating nanofibers containing biodegradable polymers obtained by electrospinning and regrowth factors on the first support and having a plurality of pores formed therein; and a third support made by accumulating nanofibers of the biodegradable polymers obtained by electrospinning on the second support and having a plurality of pores formed therein.

In the dental membrane according to an embodiment of the present invention, the diameters in size of the nanofibers of the third support may be smaller than the diameters in size of the nanofibers of the first support.

In the dental membrane according to an embodiment of the present invention, the thickness of the first support, the second support, and the third support may be gradually increased in sequence of the first to third supports.

### Advantageous Effects

According to the present invention, a dental membrane is realized by using a nanofiber web made of a biodegradable polymer, and then is decomposed a predetermined time, and thus there is an advantage of eliminating the need for a separate operation for removing the membrane.

According to the present invention, a dental membrane is prepared with a laminated structure of supports having different diameters or supports having different thicknesses of nanofibers, and controlling decomposition rates of the supports made of a biodegradable polymer, to thereby maximize the function of the membrane.

That is, in the structure in which the supports are laminated in three layers, the decomposition rate of the support closely adhered to the skin can be set quick to secure spaces in which the living tissues such as bones, alveolar bones and skins can be filled, the skeleton of the membrane may be maintained by slowing the decomposition rate of the intermediate support from among the laminated supports, and foreign matters may be prevented from penetrating into the support exposed to the outside, to thereby realize a multifunctional membrane.

Further, according to the present invention, it is possible to secure safer treatment by controlling the decomposition rate of the supports according to the function of the multifunctional membrane during completion of a dental procedure or operation (for approximately 6 months).

According to the present invention, the nanofiber web made of nanofibers has the structure most similar to the extracellular matrix (ECM) of the human body, thereby improving the skin adhesion of the dental membrane laminated with the support of the nanofiber web.

In addition, according to the present invention, the nanofibers of the supports further contain a hydrophilic agent, so that the skin adhesion of the dental membrane can be further increased.

### BRIEF DESCRIPTION OF THE DRAWINGS

FIG. 1 is a cross-sectional view of a dental membrane according to a first embodiment of the present invention.

FIG. 2 is a schematic view illustrating an electrospinning apparatus for preparing a dental membrane according to the present invention.

FIG. 3 is a schematic cross-sectional view illustrating a method of manufacturing a dental membrane according to an embodiment of the present invention.

FIG. 4 is a cross-sectional view of a dental membrane according to a second embodiment of the present invention.

FIG. 5 is a cross-sectional view of a dental membrane according to a third embodiment of the present invention.

### BEST MODE

Hereinafter, embodiments of the present invention will be described in detail with reference to the accompanying drawings.

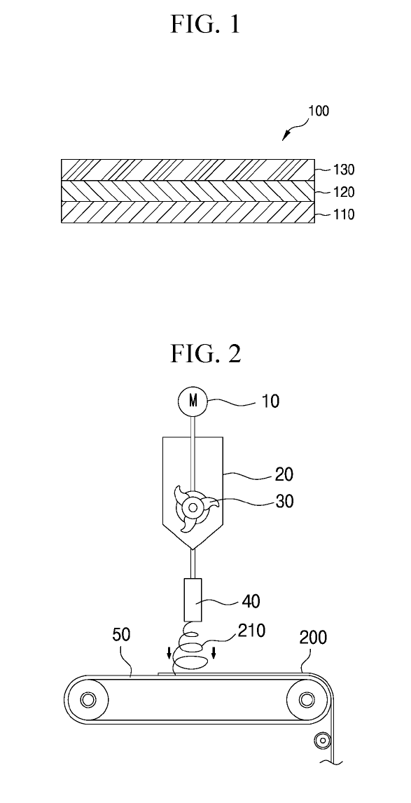

Referring to FIG. 1, a dental membrane 100 according to a first embodiment of the present invention includes: a first support 110 made by accumulating first nanofibers of a biodegradable polymer obtained by electrospinning and having a plurality of pores formed therein; a second support 120 made by accumulating second nanofibers of a biodegradable polymer obtained by electrospinning on the first support, and having a plurality of pores formed therein, in which the second nanofibers have diameters larger than the diameters of the first nanofibers; and a third support 130 made by accumulating third nanofibers of a biodegradable polymer obtained by electrospinning on the second support, and having a plurality of pores formed therein, in which the third nanofibers have diameters smaller than the diameters of the second nanofibers.

The dental membrane 100 according to the embodiment of the present invention is a dental shielding membrane that can be implanted into injured bones or periodontal tissues during implant or periodontal surgery, to help regeneration of bones and periodontal tissues, thereby preventing penetration of undesirable cells from an external environment.

That is, the dental membrane 100 according to the embodiment of the present invention has a structure in which first to third supports 110, 120 and 130 are laminated. Here, the first to third supports 110, 120 and 130 are obtained by sequentially accumulating the first to third nanofibers made of biodegradable polymers obtained by electrospinning.

The dental membrane 100 according to the embodiment of the present invention is inserted into a human oral cavity and is made of a biodegradable polymer. The dental membrane 100 is decomposed by oral secretions such as saliva secreted from the salivary glands and liquid consumed by humans, and thus there is no need for additional surgery for removing the dental membrane 100.

A biodegradable polymer is defined as a polymer that is completely decomposed into water and carbon dioxide, or water and methane gas by microorganisms such as bacteria, algae, and fungi in nature. It can be said that a biodegradable polymer is plastic whose physical and chemical structure is changed by organic matter such as bacteria in the natural world, so-called rotten plastic.

The biodegradable polymer has a characteristic that the rate of biodegradation greatly varies depending on the kind of the polymer, and the decomposition rate can be controlled according to a composition ratio of a polymer degrading relatively quickly and a polymer degrading slowly.

In particular, in the first embodiment of the present invention, the dental membrane 100 is implemented by a three-layer laminated structure of the first to third supports 110, 120 and 130 having different nanofiber diameters. The function of the membrane can be maximized by controlling the decomposition rates of the biodegradable polymers by setting different diameters of the first to third nanofibers of the first to third supports 110, 120 and 130.

That is, a thin fiber has a large specific surface area and thus has a high decomposition rate, and a thick fiber has a small specific surface area and thus a low decomposition rate.

Therefore, the second support 120 is sandwiched between the first and third supports 110 and 130. Accordingly, in order to set a low decomposition rate, it is preferable to set the second nanofibers of the second support 120 to have diameters thicker than the diameters of the first and third nanofibers of the first and third supports 110 and 130.

When the dental membrane 100 is operated, the first support 110 has the decomposition rate faster than that of the second support 120 into a region that is in close contact with the skin in the oral cavity. The diameters of the first nanofibers of the first support 110 are smaller than the diameters of the second nanofibers of the second support 120 in order to secure spaces that can be filled with living tissues such as bones, alveolar bones, and skin.

In addition, it is preferable that the diameters of the third nanofibers of the third support 130 should be smaller than those of the first and second nanofibers of the first and second supports 110 and 120 so that the third support 130 is configured to allow the membrane has the smallest pores to prevent foreign matter from penetrating into the skin.

Here, the diameters of the first and third nanofibers of the first and third supports 110 and 130 are less than 200 nm, the diameters of the second nanofibers of the second support 130 are in the range of 200 nm to 1 μm, and the second nanofibers of the second support 120 are thicker than the first and third nanofibers of the first and third supports 110 and 130.

Each of the first to third supports 110, 120, and 130 of the dental membrane 100 is prepared by forming a nanofiber web having a plurality of pores by electrospinning a spinning solution containing a mixture of a biodegradable polymer and a solvent to obtain nanofibers, and accumulating the nanofibers.

The biodegradable polymer may be one or a mixture of at least two of PLA (Poly Lactic Acid), PLLA (Poly (L-lactic acid)), PGA (Poly (glycolic acid)), PLGA (Poly (lactide-co-glycolide)), PCL (Polycaprolactone) and PDO (1,3-Propanediol).

The solvent may employ at least one selected from the group consisting of DMAc (N, N-dimethyl acetoamide), DMF (N, N-dimethylformamide), NMP (N-methyl-2-pyrrolidinone), DMSO (dimethyl sulfoxide), THF (tetra-hydrofuran), EC (ethylene carbonate), DEC (diethyl carbonate), DMC (dimethyl carbonate), EMC (ethyl methyl carbonate), PC (propylene carbonate), water, acetic acid, formic acid, chloroform, dichloromethane, acetone, and isopropylalchol.

Therefore, the nanofiber web made of the nanofibers applied to the support of the dental membrane according to some embodiments of the present invention has the structure most similar to the extracellular matrix (ECM) of the human body, thereby improving the adhesion to the skin.

In addition, in some embodiments of the present invention, when a membrane is implemented with a laminated structure of a nanofiber web having excellent flexibility, and operated, a bending operation is required to match the shape of teeth and gums. This bending operation can be performed smoothly.

FIG. 2 is a schematic view illustrating an electrospinning apparatus for preparing a dental membrane according to the present invention.

Referring to FIG. 2, an electrospinning apparatus for producing a dental membrane according to an embodiment of the present invention is characterized in that a stirring tank 20 for supplying a stirred spinning solution is connected to a spinning nozzle 40, a grounded collector 50 in the form of a conveyor that moves at a constant speed is placed in a lower portion of the electrospinning apparatus and spaced from the spinning nozzle 40, and the spinning nozzle 40 is connected to a high voltage generator.

Here, the biodegradable polymer and the solvent are mixed with a stirrer 30 driven by a motor 10 to prepare a spinning solution. Alternatively, a pre-mixed spinning solution may be used before being put into the electrospinning device without mixing a biodegradable polymer and a solvent in the stirrer 30.

Thereafter, when a high voltage electrostatic force is applied between the collector 50 and the spinning nozzle 40, the spinning solution is made into ultrafine nanofibers 210 by the spinning nozzle 40 and spun onto the collector 50, and the nanofibers 210 are accumulated on the collector 50, to thus produce a nanofiber web 200 of the support to be used for the dental membrane.

More specifically, the spinning solution discharged from the spinning nozzle 40 is discharged as the nanofibers 210 while passing through the spinning nozzle 40 charged by the high voltage generator, and the nanofibers 210 are sequentially laminated on the grounded collector 50 provided in the form of a conveyor moving at a certain speed to form the nanofiber web 200 for the dental membrane.

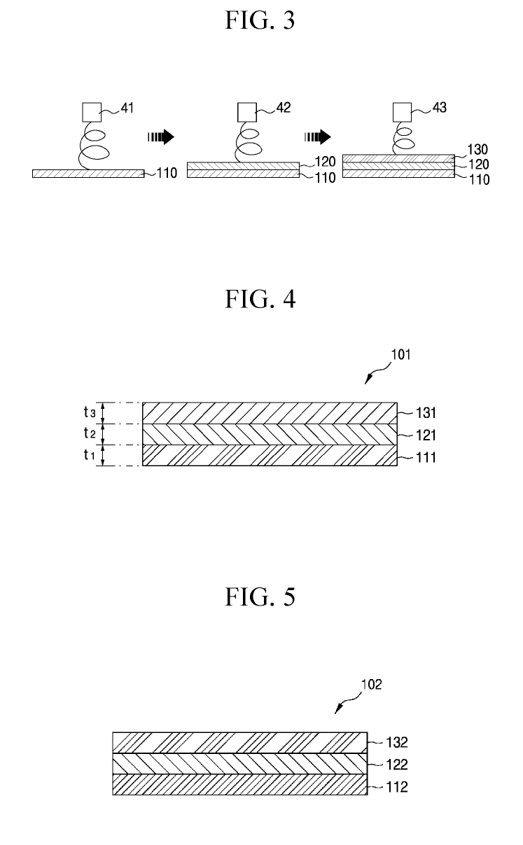

FIG. 3 is a schematic cross-sectional view illustrating a method of manufacturing a dental membrane according to an embodiment of the present invention.

The dental membrane according to the embodiment of the present invention is formed by accumulating nanofibers discharged from the first to third spinning nozzles 41, 42, and 43.

The spinning solution in which the biodegradable polymer and the solvent are mixed is supplied to the first to third spinning nozzles 41, 42 and 43 to discharge nanofibers having different diameters, and the first to third spinning nozzles 41, 42 and 43 are sequentially placed on the collector 50 moving at a constant speed of the above-described electrospinning apparatus.

When the first nanofibers are discharges from the first spinning nozzle 41 to form the first support 110 and then the first support 110 is moved to the lower portion of the second spinning nozzle 42, the second nanofibers are discharged onto the first support 110 from the second spinning nozzle 42, to thus laminate the second support 120 on the first support 110.

When a laminate of the second support 120 and the first support 110 moves to the lower portion of the third spinning nozzle 43, the third spinning nozzle 43 discharges the third nanofibers to the upper portion of the second support 120, to thus laminate the third support 130 on the second support 120.

Referring to FIG. 4, a dental membrane 101 according to a second embodiment of the present invention includes: a first support 111 made by accumulating first nanofibers of a biodegradable polymer obtained by electrospinning and having a plurality of pores formed therein; a second support 121 made by accumulating second nanofibers of a biodegradable polymer obtained by electrospinning on the first support 111, and having a plurality of pores formed therein, in which the second support 121 is thicker than the first support 111; and a third support 131 made by accumulating third nanofibers of a biodegradable polymer obtained by electrospinning on the second support 121, and having a plurality of pores formed therein, in which the third support is thicker than the first and second supports 111 and 121.

In the second embodiment of the present invention, the thickness is sequentially increased from the first support 111 to the third support 131. That is, when the first to third supports 111, 121 and 131 made of biodegradable polymers are thin, the decomposition rate is high, and when the thickness thereof is large, the decomposition rate is low.

Therefore, the first support 111 closely attached to the skin in the oral cavity is set to have the thinnest thickness t₁ to then be first decomposed, to thus secure spaces that can be filled with living tissues such as bones, alveolar bones, and skins to be regenerated. Then, the second support 121 is set to have an intermediate thickness t₂ so that the second support 121 is next decomposed. Finally, the third support 131 is set to have the thickest thickness t₃ so that the third support 131 is next decomposed.

After the dental membrane 101 according to the second embodiment of the present invention is operated for treatment, it is preferable that the first support 111 should have a thickness capable of being decomposed in 1 to 2 months, the second support 121 should have a thickness capable of being decomposed in 3 to 4 months, and the third support 131 should have a thickness capable of being decomposed in 5 to 6 months. For this, although the decomposition rate varies depending on the material of the biodegradable polymer, when the first support 111 is in contact with the growing living tissue, the thickness of the second support 121 is preferably thicker than that of the first support 111, and the thickness of the third support 131 is preferably thicker than those of the first and second supports 111 and 121.

Referring to FIG. 5, the dental membrane 102 according to the third embodiment of the present invention includes: a first support 112 made by accumulating nanofibers containing biodegradable polymers obtained by electrospinning and bone growth factors and having a plurality of pores formed therein; a second support 122 made by accumulating nanofibers containing biodegradable polymers obtained by electrospinning and regrowth factors on the first support 112 and having a plurality of pores formed therein; and a third support 132 made by accumulating nanofibers of the biodegradable polymers obtained by electrospinning on the second support 122 and having a plurality of pores formed therein.

The dental membrane 102 according to the third embodiment of the present invention contains the bone growth factors in the nanofibers of the first support 112 and the regrowth factors in the nanofibers of the second support 122, so that the dental membrane 102 can promote bone and cell growth after the procedure of the dental membrane 102.

The nanofibers of the supports included in the dental membranes of the first to third embodiments of the present invention further include a hydrophilic agent, which can increase skin adhesion. This hydrophilic agent is mixed with the biodegradable polymer and the solvent in the spinning solution, so that the nanofibers obtained by the electrospinning process contain the hydrophilic agent.

Here, the additive for hydrophilic treatment, that is, the hydrophilic agent may be one of Tween 80, Pluronic, and PVP.

While the present invention has been particularly shown and described with reference to exemplary embodiments thereof, by way of illustration and example only, it is clearly understood that the present invention is not to be construed as limiting the present invention, and various changes and modifications may be made by those skilled in the art within the protective scope of the invention without departing off the spirit of the present invention.

### INDUSTRIAL APPLICABILITY

The present invention can be applied to a dental membrane capable of controlling a decomposition rate of a laminated nanofiber web support thereby maximizing function of the dental membrane and improving skin adhesion.

What is claimed is:

1. A dental membrane comprising:
a first support made by accumulating first nanofibers of a biodegradable polymer obtained by electrospinning and having a plurality of pores formed therein;
a second support made by accumulating second nanofibers of a biodegradable polymer obtained by electrospinning on the first support, and having a plurality of pores formed therein, in which the second nanofibers have diameters larger than the diameters of the first nanofibers; and
a third support made by accumulating third nanofibers of a biodegradable polymer obtained by electrospinning on the second support, and having a plurality of pores formed therein, in which the third nanofibers have diameters smaller than the diameters of the second nanofibers.

2. The dental membrane of claim 1, wherein the diameters in size of the third nanofiber of the third support are smaller than the diameters in size of the first nanofiber of the first support.

3. The dental membrane of claim 1, wherein the diameters of the first nanofibers are less than 100 nm.

4. The dental membrane of claim 1, wherein the diameters of the third nanofibers are in the range of 200 nm to 1 μm.

5. The dental membrane of claim 1, wherein the biodegradable polymer is one or a mixture of at least two of PLA (Poly Lactic Acid), PLLA (Poly(L-lactic acid)), PGA (Poly(glycolic acid)), PLGA (Poly(lactide-co-glycolide)), PCL (Polycaprolactone) and PDO (1,3-Propanediol).

6. The dental membrane of claim 1, wherein at least one of the first to third nanofibers includes a hydrophilic agent.

7. The dental membrane of claim 6, wherein the hydrophilic agent is one of Tween 80, Pluronic and PVP.

8. The dental membrane of claim 1, wherein the first support is in contact with a living tissue to be grown, the thickness of the second support is thicker than the thickness of the first support, and the thickness of the third support is thicker than the thicknesses of the first and second supports.

9. The dental membrane of claim 8, wherein the first support has a thickness capable of being decomposed in 1 to 2 months, the second support has a thickness capable of being decomposed in 3 to 4 months, and the third support has a thickness capable of being decomposed in 5 to 6 months.

10. A dental membrane comprising:
a first support made by accumulating nanofibers containing biodegradable polymers obtained by electrospinning and bone growth factors and having a plurality of pores formed therein;
a second support made by accumulating nanofibers containing biodegradable polymers obtained by electrospinning and regrowth factors on the first support and having a plurality of pores formed therein; and
a third support made by accumulating nanofibers of the biodegradable polymers obtained by electrospinning on the second support and having a plurality of pores formed therein.

11. The dental membrane of claim 10, wherein the diameters in size of the nanofibers of the third support are smaller than the diameters in size of the nanofibers of the first support.

12. The dental membrane of claim 10, wherein the thickness of the first support, the second support, and the third support are gradually increased in sequence of the first to third supports.
